(19) United States
(12) Patent Application Publication
(10) Pub. No.: US 2011/0123617 A1
(43) Pub. Date: May 26, 2011

(54) CLIOQUINOL FOR THE TREATMENT OF HEMATOLOGICAL MALIGNANCIES

(75) Inventors: Aaron D. Schimmer, Thornhill (CA); Xinliang Mao, Markham (CA); Keith Stewart, Scottsdale, AZ (US)

(73) Assignee: University Health Network

(21) Appl. No.: 12/738,388

(22) PCT Filed: Oct. 16, 2008
(86) PCT No.: PCT/CA2008/001812

§ 371 Date: Jan. 15, 2011

Related U.S. Application Data
(60) Provisional application No. 60/980,783, filed on Oct. 18, 2007.

(51) Int. Cl. A61K 9/28; A61K 31/47; A61K 33/34; A61P 35/00; A61P 35/02

(52) U.S. Cl. 424/465; 514/311; 424/632

(57) ABSTRACT

The present invention relates to compositions and methods for treating hematological malignancies and proliferative diseases, disorders and conditions involving increased D-cyclin expression. In particular, the present invention relates to compositions and methods for treating the hematological malignancies acute myeloid leukemia (AML) and multiple myeloma (MM) using clioquinol.

### FIELD OF THE INVENTION

The invention relates to methods and compositions for the treatment of hematologic malignancies and particularly to methods and compositions for the treatment of acute myeloid leukemia (AML) and multiple myeloma (MM) in a subject.

### BACKGROUND OF THE INVENTION

Acute myeloid leukemia (AML) and multiple myeloma (MM) are hematological malignant diseases resulting in the proliferation of abnormal cells of myeloid and lymphoid origin, respectively. Both diseases are characterized by poor responses to standard therapies. For example, elderly patients with either AML or myeloma and poor risk cytogenetics have a median survival of less than one year. Thus, for these patients and those with relapsed refractory disease novel therapies are needed. As many of these patients are frail, therapies that achieve an anti-myeloma or anti-leukemia effect without significant toxicity are highly desirable.

Bortezomib, a proteasome inhibitor, has efficacy in the treatment of myeloma (16) and preliminary data has supported its evaluation for the treatment of other malignancies.

The proteasome mediates the proteasomal degradation pathway which is necessary to rid cells of excess and misfolded proteins as well as to regulate levels of proteins responsible for processes such as cell cycle progression, DNA repair and transcription (reviewed in (1)). The proteasomal degradation pathway is initiated by the sequential activity of E1, E2 and E3 enzymes that mark proteins for degradation by adding chains of ubiquitin molecules to proteins' lysine residues (reviewed in (2, 3)). Once tagged with ubiquitin, proteins are degraded by the 26S proteasome, a multimeric enzymatic complex located in the nucleus and cytoplasm. Inhibition of the proteasome induces cell death through a variety of mechanisms including accumulation of misfolded proteins and NFKB activation (4-7).

The 26S proteasome is comprised of the 19S proteasome that serves a regulatory function and the 20S proteasome that is responsible for the enzymatic degradation of proteins. The 19S proteasome is a multi-subunit complex that recognizes ubiquitin tagged proteins and then de-ubiquitinates, unfolds, and passes them to the 20S proteasome (8). Two 19S subunits cap each end of the barrel-shaped 20S proteasome (9). The 20S proteasome is comprised of alpha and beta subunits that form outer and inner rings of this complex, respectively (10, 11). The alpha subunits on the outside of the 20S proteasome give this complex its barrel shape and allow substrates to enter the center of the barrel (10, 11). The beta subunits form the inside rings of the 20S proteasome and perform the proteolytic function of the complex (11). The 20S proteasome possesses caspase-like, trypsin-like and chymotrypsin-like peptidase activity that is mediated by the β1, β2, and β5 subunits, respectively (10, 12).

A variety of synthetic and natural proteasome inhibitors have been developed and characterized. Proteasome inhibitors such as bortezomib and NPI-0052 bind threonine residues in the active sites of the β subunits of the 20S proteasome and thereby inhibit the enzymatic activity of the proteasome (13-15). The FDA-approved proteasome inhibitor bortezomib is a preferential competitive inhibitor of chymotrypsin-like activity which is the rate limiting enzyme in the proteasome (7, 12, 13, 15), whereas, Nereus pharmaceutical's drug NPI-0052 irreversibly inhibits all of the enzymes in the proteasome (7, 13, 14).

Cyclin D2 is over-expressed in multiple myeloma (MM) and in high-risk patients with acute myeloid leukemia (AML), contributing to their pathogenesis and chemoresistance (21, 22) (23).

In addition, patients with malignancies including AML have higher levels of copper in their serum compared to healthy controls. Levels of copper are higher in malignant cells compared to normal cells (17-19).

Clioquinol is a copper-binding halogenated 8-hydroxyquinoline (FIG. 1) that was used in the 1950's to 1970's as an oral anti-parasitic agent for the treatment and prevention of intestinal amebiasis, but its mechanism of action as an anti-microbial was unknown. Clioquinol has recently been shown to inhibit the proteasome in solid tumor cells such as breast and colon cancer cells through a copper-dependent mechanism (28-30). Its effect on other cancer cells is unknown.

### SUMMARY OF THE INVENTION

Described herein is a novel treatment for hematological malignancies and proliferative disorders over-expressing cyclin D. The inventors had demonstrated that clioquinol inhibits cyclin D2 transactivation and reduces cyclin D2 levels in cells over-expressing cyclin D2. The inventors have also shown that clioquinol induces cell death in hematological malignancies and reduces tumor size in an in vivo mouse model.

Accordingly, in one aspect the present application describes a method for treating a hematological malignancy comprising administering an effective amount of clioquinol to a subject in need of such treatment. In one embodiment the hematological malignancy is multiple myeloma (MM). In another embodiment, the hematological malignancy is leukemia. In a further embodiment the leukemia is acute myeloid leukemia.

A further aspect is a method of treating acute myeloid leukemia or multiple myeloma comprising administering an effective amount of clioquinol to a subject in need of such treatment.

Another aspect provides a method of treating a proliferative disease, disorder or condition involving increased cyclin D expression comprising administering an effective amount of clioquinol to a subject in need of such treatment.

In certain embodiments, the effective amount is within the range of 1 to 200 mg/kg body weight. In one embodiment, the effective amount is within the range of 5 to 50 mg/kg body weight.

In certain aspects, the application describes a method of treating hematological malignancies such as multiple myeloma (MM) and leukemia including acute myeloid leukemia (AML), comprising contemporaneously administering clioquinol and a copper compound.

In a further aspect, the application describes methods wherein the clioquinol administered is comprised in a composition described herein.

Another aspect is a use of clioquinol for the treatment of a hematological malignancy.

A further aspect is a use of clioquinol in the preparation of a medicament for the treatment of a hematological malignancy.

In certain embodiments, the use of clioquinol is for the treatment of multiple myeloma. In other embodiments the use of clioquinol is for the treatment of leukemia. In yet other embodiments, the use of clioquinol is for the treatment of acute myeloid leukemia.

Another aspect is a use of clioquinol for the treatment of a proliferative disease, disorder, or condition involving increased cyclin D expression. In certain embodiments, the proliferative disease, disorder, or condition involves increased cyclin D2 expression.

A further aspect is a use of clioquinol in the preparation of a medicament for the treatment of a proliferative disease disorder or condition involving increased cyclin D expression.

Yet another aspect is a use of clioquinol for the treatment of acute myeloid leukemia or multiple myeloma.

Another aspect is a use of clioquinol in the preparation of a medicament for treatment of acute myeloid leukemia or multiple myeloma.

An additional aspect is the use of clioquinol in the preparation of a medicament for treatment of acute myeloid leukemia or multiple myeloma, wherein the effective amount of clioquinol is within the range of 1 to 200 mg/kg body weight, suitably in the range of 5 to 50 mg/kg body weight.

A further aspect is a use of a therapeutically effective amount of a composition comprising clioquinol for treating a hematological malignancy. Another aspect is a use of a therapeutically effective amount of a composition comprising clioquinol for treating a proliferative disease, disorder or condition involving increased cyclin D expression.

A further aspect is a pharmaceutical composition for the treatment of a hematological malignancy comprising clioquinol and a pharmaceutically acceptable carrier in a dosage form, wherein the dosage form is suitable for oral administration or injection.

Another aspect is a pharmaceutical composition for the treatment of a proliferative disease, disorder or condition involving increased cyclin D expression.

A further aspect of the invention is a pharmaceutical composition for the treatment of a hematological malignancy or of a proliferative disease, disorder or condition involving increased cyclin D expression wherein a solid dosage form contains from about 20 mg to about 1000 mg clioquinol, suitably from about 50 mg to about 500 mg clioquinol.

A further aspect is a pharmaceutical composition for the treatment of a hematological malignancy or of a proliferative disease, disorder or condition involving increased cyclin D expression wherein the dosage form is a liquid dosage form that contains from about 20 mg to about 2000 mg clioquinol, suitably from about 40 mg to about 500 mg clioquinol.

A further aspect is a pharmaceutical composition for treatment of acute myeloid leukemia or multiple myeloma in a subject, which composition comprises as active ingredient clioquinol and a pharmaceutically acceptable carrier in unit dosage form, wherein the pharmaceutical composition is suitable for oral administration or injection.

A further aspect is a pharmaceutical composition for the treatment of a hematological malignancy or of a proliferative disease, disorder or condition involving increased cyclin D expression comprising clioquinol and a pharmaceutically acceptable carrier in unit dosage form in an amount suitable to provide 1 to 200 mg of clioquinol/kg body weight, suitably 5 to 50 mg of clioquinol/kg body weight, formulated into a solid oral dosage form, a liquid dosage form, or an injectable dosage.

A further aspect is a composition, wherein the amount of clioquinol is an effective amount for treatment of acute myeloid leukemia or multiple myeloma.

A further aspect is a composition, wherein the oral dosage form is selected from enteric coated tablets, caplets, gelcaps, and capsules, comprising from 20 to less than 1000 mg, suitably from 50 to 500 mg, of clioquinol.

A further aspect is a composition, wherein the tablets or capsules containing 20 to less than 1000 mg, suitably from 50 to 500 mg, of clioquinol.

A further aspect is a commercial package comprising a composition according to the present invention, and associated therewith instructions for the use thereof for treatment of a hematological malignancy such as acute myeloid leukemia or multiple myeloma in a subject in need of such treatment.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

An embodiment of invention will now be described in relation to the drawings in which:

FIG. 1 is a series of scans of immunoblots and graphs demonstrating that clioquinol decreases cyclin D2, increases p21 and 27, and arrests cells in G0/G1 phase. A) Effect of increasing concentrations of clioquinol on LP1 and MY5 multiple myeloma cells at twenty hours after clioquinol treatment; determined by SDS-PAGE immunoblotting. B) Effect of increasing concentrations of clioquinol on the G0/G1 ratio in AML3, U937, LP1 and MY5 cells at twenty hours after clioquinol treatment; determined by PI staining and flow cytometry.

FIG. 2 is a series of scans of immunoblots demonstrating that clioquinol increases the abundance of ubiquitinated proteins. Effect of increasing concentrations of clioquinol on LP1 and MY5 multiple myeloma cells at twenty hours after clioquinol treatment; determined by SDS-PAGE immunoblotting.

FIG. 3 is a series of graphs demonstrating the effect of increasing concentrations of clioquinol on proteasome activity of MDAY-D2 and JJN3 cells. A) Proteasome activity of MDAY-D2 and JJN3 cellular extracts after two hours of treatment with clioquinol (CQ), CuCl₂ (Cu), or an equimolar concentration of CQ and Cu. After treatment, the preferential chymotrypsin-like substrate Suc-LLVY-AMC was added and the rate of free AMC was measured over time with a florescent spectrophotometric plate reader (excitation=380 nm, emission=460 nm). B) Proteasome activity of intact MDAY-D2 and JJN3 cells after two hours of treatment with clioquinol (CQ), CuCl₂ (Cu), or an equimolar concentration of CQ and Cu. After treatment, the preferential chymotrypsin-like substrate Suc-LLVY-AMC was added and the rate of free AMC was measured over time with a florescent spectrophotometric plate reader (excitation=380 nm, emission=460 nm).

FIG. 4 is a series of graphs demonstrating clioquinol inhibits the protease in primary AML cells preferentially over normal hematopoietic cells. Proteasome activity of primary acute myeloid leukemia (AML) blasts or normal peripheral blood stem cells (PBSC) after twenty hours of treatment with clioquinol (CQ), or an equimolar concentration of CO and CuCl₂ (Cu). After treatment, the preferential chymotrypsin-like substrate Suc-LLVY-AMC was added and the rate of free AMC was measured over time with a florescent spectrophotometric plate reader (excitation=380 nm, emission=460 nm).

FIG. 5 is a series of graphs demonstrating that clioquinol reduces the viability of leukemia and myeloma cells and primary AML patient samples. Cell viability of leukemia, myeloma, solid tumor and primary cells forty-eight hours after clioquinol treatment by MTS assay.

FIG. 6 is a graph demonstrating that clioquinol reduces the viability of MY5 cells after twenty-four hours of treatment with clioquinol (CQ) with or without CuCl₂ (Cu) (20 μM), and/or the copper chelator tetrathiomoylbdate (TM) (20 μM). Apoptosis was measured by Annexin V and PI staining and flow cytometry.

FIG. 7 is a series of graphs that shows treatment with clioquinol delays tumor growth in a mouse model of leukemia.

### DETAILED DESCRIPTION OF THE INVENTION

### I. Method/Uses of Clioquinol

The inventors have identified a novel therapeutic for treating hematological malignancies such as multiple myeloma (MM) and acute myeloid leukemia (AML). Using a chemical biology screen for inhibitors of cyclin D2 transactivation, the inventors have surprisingly identified the anti-parasitic compound clioquinol as being an inhibitor of cyclin D2 transactivation. Furthermore, the inventors have demonstrated that treatment with clioquinol increases expression of p21 and p27 as well as the abundance of ubiquitinated proteins in AML and MM cells. Clioquinol treatment also inhibits the chymotrypsin-like enzymatic activity of the proteasome in cell extracts and intact AML and MM cells and clioquinol-mediated inhibition of the proteasome in AML and MM cells is copper-dependent. Clioquinol induces apoptosis in myeloma and leukemia cells through a copper-dependent mechanism and dramatically reduces tumor weight in an in vivo mouse model of leukemia.

Accordingly, the present application describes a method of treating hematological malignancies including leukemia and myeloma by administering an effective amount of clioquinol to a subject in need of such a treatment.

The present application also includes the use of clioquinol for the treatment of a hematological malignancy such as a leukemia or myeloma.

The present application further describes the use of clioquinol in the preparation of a medicament for treatment of a hematological malignancy such as leukemia or multiple myeloma.

In one embodiment the hematological malignancy is a leukemia such as acute myeloid leukemia. In another embodiment the hematological malignancy is multiple myeloma.

Without wishing to be bound by theory, the mechanism of clioquinol action may involve one or more of the following pathways. As mentioned, D-cyclins are over-expressed in multiple myeloma (MM) and a subset of high-risk patients with acute myeloid leukemia (AML), contributing to their pathogenesis and chemoresistance (21, 22) (23). Further inhibition of the proteosome by Bortezomib, a protease inhibitor has been shown to have efficacy in the treatment of multiple myeloma. The proteasomal degradation pathway is necessary to rid cells of excess and misfolded proteins as well as regulate levels of proteins responsible for processes such as cell cycle progression, DNA repair and transcription (reviewed in (1)). In addition, patients with malignancies including AML have higher levels of copper in their serum. As the inventors have shown that clioquinol reduces D-cyclin expression, inhibits the proteasome and as clioquinol is a known copper binding compound, clioquinol may be affecting one or more of these pathways. Furthermore, as the inventors have demonstrated that clioquinol reduces D-cyclin expression and reduces viability of D-cyclin over-expressing cells, in one embodiment the application describes treating a proliferative disease involving increased D-cyclin expression.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with early stage myeloma can be treated to prevent progression or alternatively a subject in remission can be treated with a compound or composition described herein to prevent recurrence. Treatment methods comprise administering to a subject a therapeutically effective amount of a compound described in the present application and optionally consists of a single administration, or alternatively comprises a series of applications. For example, the compounds described herein may be administered at least once a week. However, in another embodiment, the compounds may be administered to the subject from about one time per week to about once daily for a given treatment. In another embodiment, the compound is administered twice daily. The length of the treatment period depends on a variety of factors, such as the severity of the disease, the age of the patient, the concentration, the activity of the compounds described herein, and/or a combination thereof. It will also be appreciated that the effective dosage of the compound used for the treatment or prophylaxis may increase or decrease over the course of a particular treatment or prophylaxis regime. Changes in dosage may result and become apparent by standard diagnostic assays known in the art. In some instances, chronic administration may be required.

The dosage administered will vary depending on the use and known factors such as the pharmacodynamic characteristics of the particular substance, and its mode and route of administration, age, health, and weight of the individual recipient, nature and extent of symptoms, kind of concurrent treatment, frequency of treatment, and the effect desired. Dosage regime may be adjusted to provide the optimum therapeutic response.

As used herein, the phrase “effective amount” or “therapeutically effective amount” means an amount effective, at dosages and for periods of time necessary to achieve the desired result. For example in the context or treating a hematological malignancy, an effective amount is an amount that for example induces remission, reduces tumor burden, and/or prevents tumor spread or growth compared to the response obtained without administration of the compound. Effective amounts may vary according to factors such as the disease state, age, sex, weight of the animal. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

The term “subject” as used herein includes all members of the animal kingdom including mammals, and suitably refers to humans.

In one embodiment, the subject has leukemia. In another embodiment, the subject has acute myeloid leukemia. In another embodiment, the subject has high-risk acute myeloid leukemia. In another embodiment, the subject has multiple myeloma. In a further embodiment, the subject has a refractory malignancy.

The term “hematological malignancy” as used herein refers to cancers that affect blood and bone marrow.

The term “leukemia” as used herein means any disease involving the progressive proliferation of abnormal leukocytes found in hemopoietic tissues, other organs and usually in the blood in increased numbers. Leukemia includes acute myeloid leukemia.

The term “myeloma” as used herein means any tumor or cancer composed of cells derived from the hemopoietic tissues of the bone marrow. For example, myeloma includes multiple myeloma.

The term “proliferative disease involving increased expression of cyclin D” means any disease where a cell type increases in numbers and has increased expression of cyclin D. Three D-cyclins are known including cyclin D1, cyclin D2 and cyclin D3. In certain embodiments the cyclin D that has increased expression is cyclin D2. One skilled in the art would readily understand that cyclin D expression is easily detected by methodologies known in the art such as protein detection methods such as immunoblotting and ELISA and nucleic acid methods such as RT-PCR and northern analysis. Increased cyclin D expression can be determined by comparing the level of cyclin D expression to one or more control samples, individually or pooled.

The inventors have also found that a copper compound can enhance the therapeutic effect of clioquinol. Accordingly in certain aspects, the methods comprise contemporaneous administration of clioquinol and a copper compound. In one embodiment the copper compound is a copper salt. In another embodiment the copper compound is copper oxide. In certain embodiments the copper compound is administered, contemporaneously as an oral tablet or capsule.

As used herein, “contemporaneous administration” and “administered contemporaneously” means that two substances are administered to a subject such that they are both biologically active in the subject at the same time. The exact details of the administration will depend on the pharmacokinetics of the two substances in the presence of each other, and can include administering one substance within 24 hours of administration of the other, if the pharmacokinetics are suitable. Designs of suitable dosing regimens are routine for one skilled in the art. In particular embodiments, two substances will be administered substantially simultaneously, i.e. within minutes of each other, or in a single composition that comprises both substances.

Further, the inventors have also demonstrated that clioquinol inhibits cyclin D expression. Accordingly, the application describes a method of inhibiting cyclin D expression in a cell or in a subject, comprising administering clioquinol to the cell or subject.

Inhibiting cyclin D expression means in one embodiment, reducing expression by at least 50%, at least 60%, at least 70%, at least 80%, at least 90% or at least 95% as determined using assays known in the art, for example immunoblotting.

As used herein, to “inhibit” or “suppress” or “reduce” a function or activity, such as proteasomal activity, is to reduce the function or activity when compared to otherwise same conditions except for a condition or parameter of interest, or alternatively, as compared to another condition. Similarly to “inhibit” or “suppress” or “reduce” expression such as cyclin D expression, is to reduce the level of expression when compared to otherwise same condition or parameter or interest, or alternatively as compared to another condition. The terms “inhibitor” and “inhibition”, in the context of the present application, are intended to have a broad meaning and encompass clioquinol which directly or indirectly (e.g., via reactive intermediates, metabolites and the like) acts on the proteasome, and/or decrease cyclin D expression.

The inventors have also demonstrated that clioquinol induces cell death in leukemia and myeloma cells. Accordingly, the application describes a method of inducing cell death in a leukemia cell or a myeloma cell comprising administering clioquinol. In certain embodiments, a copper compound is administered contemporaneously to induce cell death.

The term “cell death” as used herein includes all forms of cell death including necrosis and apoptosis.

Inhibiting proteasomal activity means reducing proteasomal activity by at least 50%, at least 60%, at least 70%, at least 80%, at least 90% or at least 95% as determined using a proteasomal activity assay known in the art. For example proteasomal activity of an extract or sample can be determined by assaying the rate of free AMC released from the proteasomal substrate Suc-LLVY-AMC as described herein.

The “proteasome” as used herein refers to a multimeric enzymatic complex involved in the degradation of protein. The proteasome comprises multiple protease activities including a chymotrypsin-like protease activity. A mentioned, the proteasomal degradation pathway is necessary to rid cells of excess and misfolded proteins as well as regulate levels of proteins responsible for processes such as cell cycle progression, DNA repair and transcription (reviewed in (1)).

The term “proteasomal activity” as used herein refers to an activity of the proteasome and “chymotrypsin-like proteasomal activity” refers to the protease activity of the proteasome that is specific for chymotrypsin or chymotrypsin-like substrates

### II. Compositions

The application also describes compositions comprising clioquinol for the treatment of hematological malignancies or proliferative disorders involving increased cyclin D expression.

The term “clioquinol” as used herein means 5-chloro-7-iodo-8-hydroxyquinoline and includes all pharmaceutically acceptable salts, solvates, and prodrugs thereof as well as combinations thereof. Clioquinol is also known as iodochlorhydroxyquin.

It is to be clear that the present application describes pharmaceutically acceptable salts, solvates and prodrugs of clioquinol and mixtures comprising two or more of clioquinol, pharmaceutically acceptable salts of clioquinol, pharmaceutically acceptable solvates of clioquinol and prodrugs of clioquinol.

The compounds may have at least one asymmetric centre. Where the compounds described herein possess more than one asymmetric centre, they may exist as diastereomers. It is to be understood that all such isomers and mixtures thereof in any proportion are encompassed within the scope of the present invention. It is to be understood that while the stereochemistry of the compounds of the invention may be as provided for in any given compound listed herein, such compounds of the invention may also contain certain amounts (e.g. less than 20%, suitably less than 10%, more suitably less than 5%) of compounds of the invention having alternate stereochemistry.

The term “pharmaceutically acceptable” means compatible with the treatment of animals, in particular, humans.

The composition may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylarnino ethanol, histidine, procaine, etc.

The term “pharmaceutically acceptable salt” means an acid addition salt which is suitable for or compatible with the treatment of patients.

The term “solvate” as used herein means clioquinol or a pharmaceutically acceptable salt of clioquinol, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates of the compounds of the invention will vary depending on the compound and the solvate. In general, solvates are formed by dissolving the compound in the appropriate solvent and isolating the solvate by cooling or using an antisolvent. The solvate is typically dried or azeotroped under ambient conditions.

Compositions include clioquinol prodrugs. In general, such prodrugs will be functional derivatives of clioquinol which are readily convertible in vivo into the compound from which it is notionally derived. Prodrugs of clioquinol may be conventional esters formed with the available hydroxy. For example, the available OH in clioquinol may be acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, the prodrugs of the compounds of the invention are those in which one or more of the hydroxy groups in the compounds is masked as groups which can be converted to hydroxy groups in vivo. Conventional procedures for the selection and preparation of suitable prodrugs are described, for example, in “Design of Prodrugs” ed. H. Bundgaard, Elsevier, 1985.

Clioquinol is suitably formulated into pharmaceutical compositions for administration to human subjects in a biologically compatible form suitable for administration in vivo. Accordingly, the present invention further includes a pharmaceutical composition comprising clioquinol and a pharmaceutically acceptable carrier and/or diluent.

The application in one aspect, also describes a pharmaceutical composition comprising an effective amount of clioquinol and a pharmaceutically acceptable carrier for treatment of a leukemia or multiple myeloma in a subject in need of such treatment.

The compositions described herein can be prepared by per se known methods for the preparation of pharmaceutically acceptable compositions that can be administered to subjects, such that an effective quantity of the active substance is combined in a mixture with a pharmaceutically acceptable vehicle. Suitable vehicles are described, for example, in Remington's Pharmaceutical Sciences. On this basis, the compositions include, albeit not exclusively, solutions of the substances in association with one or more pharmaceutically acceptable vehicles or diluents, and contained in buffered solutions with a suitable pH and iso-osmotic with the physiological fluids.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the patient.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesyl-phosphotidyl-ethanolamine (DOPE), and liposomes. Such compositions should contain a therapeutically effective amount of the compound, together with a suitable amount of carrier so as to provide the form for direct administration to the patient.

The compositions described herein can be administered for example, by parenteral, intravenous, subcutaneous, intramuscular, intracranial, intraorbital, ophthalmic, intraventricular, intracapsular, intraspinal, intracisternal, intraperitoneal, intranasal, aerosol or oral administration.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Wherein the route of administration is oral, the dosage form may be for example, incorporated with excipient and used in the form of enteric coated tablets, caplets, gelcaps, capsules, ingestible tablets, buccal tablets, troches, elixirs, suspensions, syrups, wafers, and the like. The dosage form may be solid or liquid.

Accordingly in one embodiment, the application describes a pharmaceutical composition wherein the dosage form is a solid dosage form.

The term “solid dosage form” is to be understood to refer to individually coated tablets, capsules, granules or other non-liquid dosage forms suitable for oral administration. It is to be understood that the solid dosage form includes, but is not limited to, non-controlled release, controlled release and time-controlled release dosage form units, employed suitably in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet.

Compositions suitable for buccal or sublingual administration include tablets, lozenges, and pastilles, wherein the active ingredient is formulated with a carrier such as sugar, acacia, tragacanth, or gelatin and glycerin.

In another embodiment the application describes a pharmaceutical composition wherein the dosage form is a liquid dosage form.

The term “liquid dosage form” is to be understood to refer to non-solid dosage forms suitable for, but not limited to, intravenous, subcutaneous, intramuscular, or intraperitoneal administration. Solutions of clioquinol described herein can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999.

Sustained or direct release compositions can be formulated, e.g. liposomes or those wherein the active compound is protected with differentially degradable coatings, such as by microencapsulation, multiple coatings, etc. It is also possible to freeze-dry the compounds of the invention and use the lypolizates obtained, for example, for the preparation of products for injection.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

In one embodiment the dosage form comprises about 20 mg to 2000 mg of clioquinol. In another embodiment, the dosage form comprises about 50 mg to 500 mg of clioquinol. The dosage form may alternatively comprise 40 to 500 mg, 250 to 500 mg, 1 to 200 mg of clioquinol/kg body weight, 5 to 50 mg of clioquinol/kg body weight, 10 to 40 mg of clioquinol/kg body weight or 25 mg of clioquinol/kg body weight of a subject in need of such treatment.

Also included are methods of treating a proliferative disease involving increased cyclin D expression or a hematological malignancy such as AML or MM and administering an effective amount of one of the pharmaceutical compositions described herein to a subject in need of such treatment.

Another aspect provides a commercial package comprising a composition described herein, and associated therewith instructions for the use thereof for treatment of a hematological malignancy such as acute myeloid leukemia or multiple myeloma or a proliferative disease, disorder or condition involving increased cyclin D2 expression in a subject in need of such treatment. In another embodiment, a commercial package is provided comprising a composition described herein, and associated therewith instructions for the use thereof for inhibiting cyclin D2 expression. Another embodiment provides a commercial package comprising a composition described herein, and associated therewith instructions for the inducing cell death in a myeloma or a leukemia cell.

The following non-limiting examples are illustrative of the present invention:

### EXAMPLES

### Materials and Methods

### Cell Culture, Constructs and Transduction

Mouse fibroblast NIH3T3 cells were maintained in Dulbeco's Modified Eagle's medium plus 10% calf serum (Hyclone, Logan, Utah). Myeloma cell lines and leukemia cell lines were grown in Iscove's modified essential medium (IMEM) plus 10% fetal bovine serum (FBS) (Hyclone, Logan, Utah). All the media were supplemented with 1 mM glutamate and antibiotics. Cells were cultured at 37° C. with 5% CO₂ in a humid incubator.

Full-length c-maf cDNA was subcloned into an IRES-GFP-MIEV retroviral vector. NIH3T3 cells were infected with this construct and stable cells expressing GFP and c-maf were selected by flow cytometry and immunoblotting, respectively. The full-length c-maf was also subcloned into a pcDNA3.1 vector under the control of a CMV promoter.

The promoter of cyclin D2 (−894 to −4), containing c-maf responsive element sequence (MARE), was cloned from HeLa cell genomic DNA and subcloned into the pGL2 luciferase reporter vector (Promega, Madison, Wis.). This construct was co-transfected with pcDNA3.1 containing a neomycin resistance gene into NIH3T3 wild type cells and NIH3T3 cells stably over-expressing c-maf-IRES-GFP. Cells stably expressing c-maf, GFP, and luciferase were selected for further application.

### High Throughput Screen for Inhibitors of Cyclin D2 Transactivation

NIH3T3 cells stably expressing c-maf and the cyclin D2 promoter driving luciferase (13,000 cells per well) were plated in 96-well plates by the Biomek FX liquid handler (Beckman, Fullerton, Calif.). The same workstation was used for plate formatting and reagent distribution. After the cells had adhered (6 hr after plating), they were treated with aliquots of molecules from LOPAC (Sigma, St. Louis, Mo.) and Prestwick (Prestwick Chemical Inc, Illkirch, France) libraries. Final concentration of LOPAC compounds was 5 μM (0.05% DMSO) while for the Prestwick library, 10 ng of each sample was added, resulting in an average final concentration of approximately 5 μM (0.1% DMSO). Control wells, treated with vehicle alone containing consistent levels of DMSO, were distributed in the first and last columns of the plate to monitor signal variability. Cells were incubated with the molecules at 37° C. for 20 hours. After incubation, cyclin D2 transactivation was assessed by the luciferase assay and viability was assessed by the MTS assay.

### Luciferase Assay

Luciferase activity was assessed according to the manufacturer's instructions (Promega, Madison, Wis.). Briefly, the cell culture medium was removed using an EMBLA plate washer (Molecular Devices, Sunnyvale, Calif.) and 1× Glo Lysis buffer (Promega) was added by the robotic liquid handler. After 10 min incubation, an equal volume of Bright-Glo Luciferase substrate (Promega) was added and the luminescence signal was detected with a 96-well Luminoskan luminescence plate reader (Thermo Labsystem, Waltham, Mass.) with a 5 second integration.

### Cell Viability

Cell viability was assessed with the CellTiter96® Aqueous Non-Radioactive Assay kit according to manufactures' instructions (Promega). Apoptosis was measured by flow cytometry to detect cell surface Annexin V expression and propidium iodide (PI) uptake (Biovision, Mountain View, Calif., USA) as previously described (20)

### Immunoblotting

To prepare cytosolic extracts, NIH3T3 cells and myeloma cells were washed with phosphate-buffered saline (PBS, pH 7.4) and suspending in lysis buffer [10 mM Tris (pH 7.4), 150 mM NaCl, 0.1% Triton X-100, 0.5% sodium deoxycholate, and 5 mM EDTA] containing protease inhibitors (Complete tablets, Roche, Indianapolis, Ind.). Protein concentrations were determined by the Bradford assay. Immunoblot assays were performed by subjecting equal amounts of protein to SDS-PAGE gels followed by transfer to Nitrocellulose membranes. Membranes were probed with polyclonal rabbit anti-human cyclin D2 (0.5 μg/ml, both from Santa Cruz Biotech, Santa Cruz, Calif.), or monoclonal mouse-anti human p21 (1:200 v/v, Santa Cruz Biotech), monoclonal mouse-anti human p27 (1:2,500 v/v BD Transduction Laboratories), polyclonal mouse-anti human ubiquitin (1:2,000 v/v Calbiochem) and monoclonal mouse-anti-β-actin (1:10,000 v/v) (Sigma, St. Lois, Mo.). Secondary antibodies (Amersham Bioscience UK, Little Chalfont, England) were horseradish peroxidase (HRP)-conjugated goat anti-mouse IgG (1:10,000 v/v) and anti-rabbit (1:5,000 v/v). Detection was performed by the Enhanced Chemical Luminescence (ECL) method (Pierce, Rockford, Ill.).

### Cell Cycle Analysis

Cells were harvested, washed with cold PBS, suspended in 70% cold ethanol and incubated overnight at −20° C. Cells were then treated with 100 ng/ml of DNase-free RNase (InvitroGen) at 37° C. for 30 min, washed with cold PBS, and resuspended in PBS with 50 μg/ml of propidium iodine. DNA content was analyzed by flow cytometry (FACSCalibur, Becton Dickinson, Fla., USA). The percentage of cells in each phase of the cell cycle was calculated with ModiFit software (Becton Dickinson).

### Proteasome Activity

Cellular proteins were extracted from cells with lysis buffer (50 mM HEPES pH 7.5, 150 mM NaCl, 2 mM ATP, and 1% Triton X-100). The chymotrypsin-like activity of the proteasome was measured by incubating equal amounts of protein with the preferential chymotrypsin-like substrate Suc-LLVY-AMC in assay buffer (50 mM Tris-HCl, pH 7.5, 150 mM NaCl) for two hours. After incubation, was added and the rate of free AMC was measured over time with a florescent spectrophotometric plate reader (excitation=380 nm, emission=460 nm).

### In Vivo Studies

DBA-2 mice were injected intraperitoneally with MDAY-D2 murine leukemia cells. Mice were then treated twice daily by oral gavage with clioquinol (100 mg/kg) dissolved in intralipid or intralipid control. Ten days after treatment, mice were sacrificed, the intraperitoneal tumor excised, and the weight and volume of the tumor measured.

A High Throughput Screen Identifies c-maf Dependent and Independent Inhibitors of the Cyclin D2 Promoter

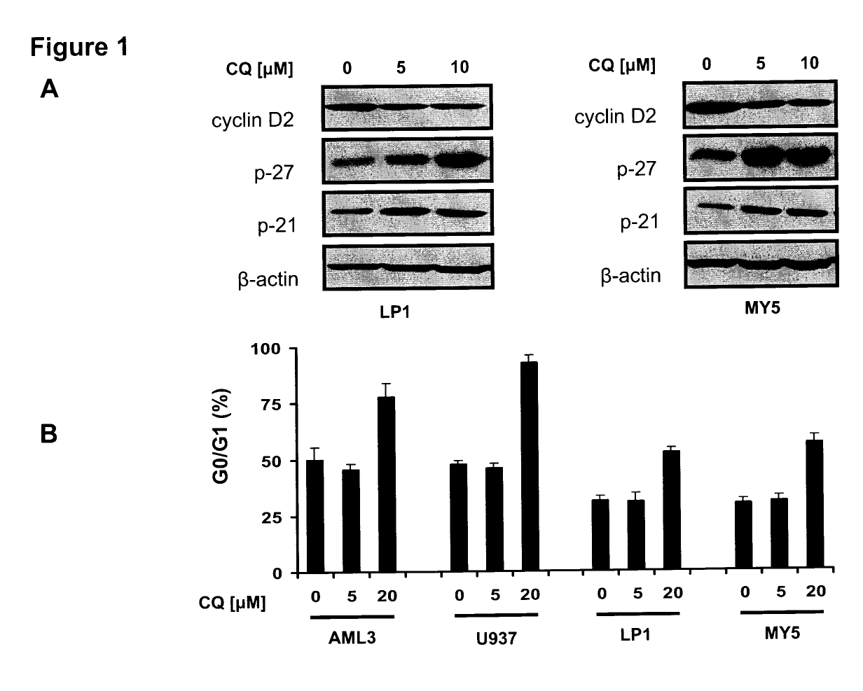

The inventor's interest in clioquinol developed after identifying this compound in a high throughput screen for c-maf dependent and independent inhibitors of the cyclin D2 promoter. Cyclin D2 is over-expressed in multiple myeloma (MM) and a subset of high-risk patients with acute myeloid leukemia (AML), contributing to their pathogenesis and chemoresistance (21, 22) (23). One of the regulators of cyclin D2 is the oncogene c-maf that is also frequently over-expressed in MM (24). Therefore, the inventors sought to identify c-maf dependent and independent inhibitors of cyclin D2. To identify such small molecule inhibitors, the inventors developed a high throughput chemical genomics screen. NIH 3T3 cells stably over-expressing a c-maf-IRES-GFP cassette in an MIEV vector and the cyclin D2 promoter (−894 by to −4 bp) driving firefly luciferase were seeded in 96 well plates and treated with aliquots of the LOPAC (1280 compounds) and Prestwick (1120 compounds) libraries of off-patent drugs and chemicals. Compounds were tested at a final concentration of ˜5 μM in <0.01% DMSO. Sixteen hours after the addition of the compounds, luciferase expression was measured as a marker of cyclin D2 transactivation. From this screen, the inventors identified both c-maf dependent (X Mao, et al, Blood in press) and c-maf independent inhibitors of the cyclin D2 promoter. The latter included the off-patent antimicrobial agent clioquinol. Additional studies demonstrated that clioquinol reduced levels of cyclin D2 in myeloma and leukemia cells, arrested cells in the G1 phase and increased expression of p21 and p27 at low micromolar concentrations (FIG. 1).

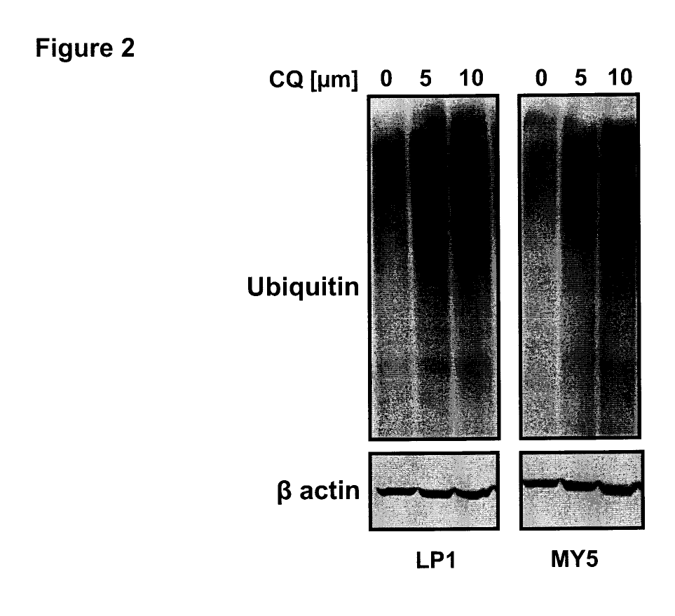

G1 cell cycle arrest, decreased cyclin D2 expression, and increased p21 and p27 expression have been associated with inhibition of the proteasome (6, 25-27). Moreover, clioquinol has recently been shown to inhibit the proteasome in breast and colon cancer cells through a copper-dependent mechanism (28-30). Therefore, the inventors investigated the effects of clioquinol on the proteasome in their system. By immunoblotting, the inventors demonstrated that treatment of leukemia and myeloma cells with clioquinol increased the amount of ubiquitinated protein, consistent with inhibition of the proteasome (FIG. 2).

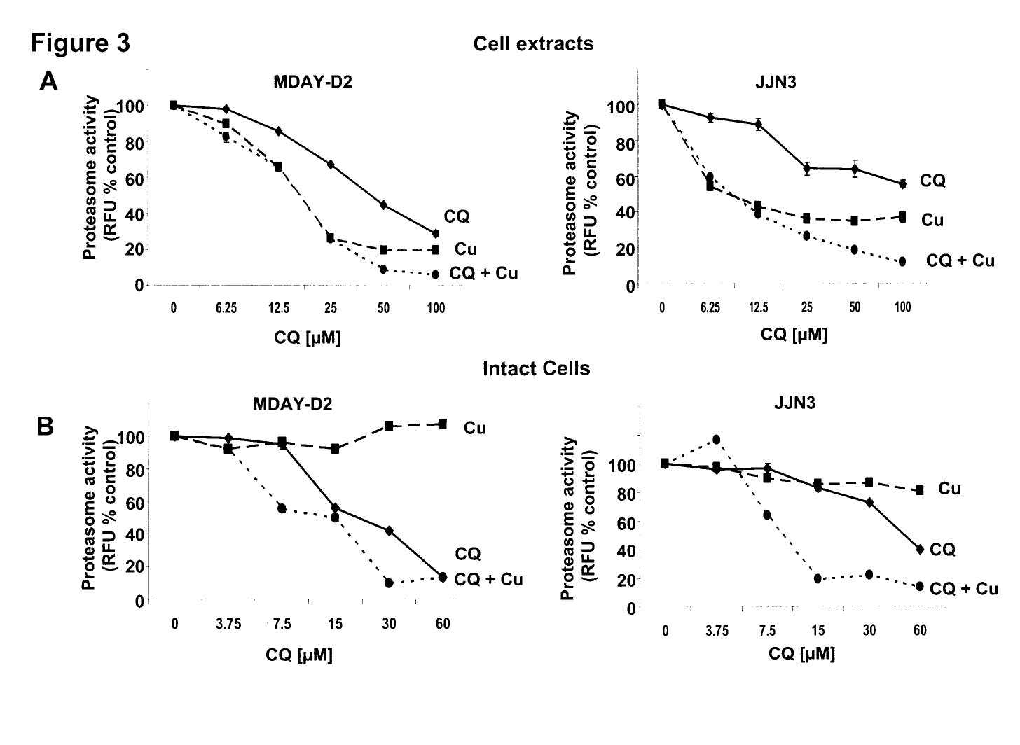

Given the above results, the inventors examined the effects of this drug on the activity of the proteasomal enzymes. Cell lysates were prepared from leukemia and myeloma cell lines and treated with increasing concentrations of clioquinol, copper, and equimolar amounts of copper and clioquinol. The chymotrypsin-like activity of the proteasome was measured by monitoring the rate of cleavage of the fluorescent substrate Suc-LLVY-AMC. Both clioquinol and copper inhibited the rate of Suc-LLVY-AMC cleavage, and the combination of clioquinol and copper produced slightly greater inhibition than copper alone (FIG. 3).

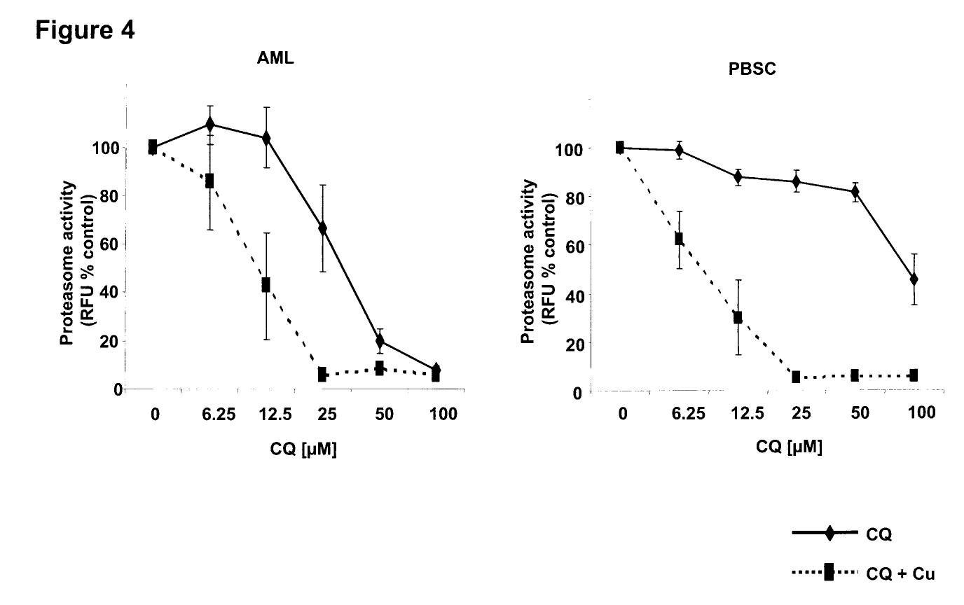

Next, the inventors examined the effect of clioquinol and copper on the function of the proteasome when added to intact cell lines and cell extracts. Leukemia and myeloma cell lines were incubated with increasing concentrations of clioquinol, copper, and equimolar concentrations of clioquinol and copper. After incubation, cell lysates were prepared and the chymotrypsin-like activity of the proteasome was measured as above. clioquinol inhibited chymotrypsin-like activity at low micromolar concentrations and at concentrations associated with the reductions in cyclin D2, G1 arrest, and increased p21 and p27 expression (FIG. 3). The addition of copper alone to intact cells had no effect on the function of proteasome. However, the addition of copper enhanced clioquinol-mediated inhibition of the proteasome (FIG. 4).

The inventors examined the effect of clioquinol and copper on primary AML cells and normal hematopoietic cells. Primary AML were obtained from the peripheral blood of patients with AML. Normal hematopoietic stem cells (PBSC) were obtained from the peripheral blood of volunteers donating stem cells for allotransplant. The mononuclear cells were isolated by Ficol separation and treated with clioquinol, copper and an equimolar concentration of clioquinol and copper. After incubation, cell lysates were prepared and the enzymatic activity of the proteasome measured as above. Clioquinol inhibited the proteasome in primary AML cells, but had no effect on normal hematopoietic cells. Interestingly, adding copper to the primary cells enhanced the activity of clioquinol in both AML and normal hematopoietic cells, but negated the differential inhibition between malignant and normal cells (FIG. 4).

### Clioquinol Induces Cell Death in Leukemia and Myeloma Cell Lines

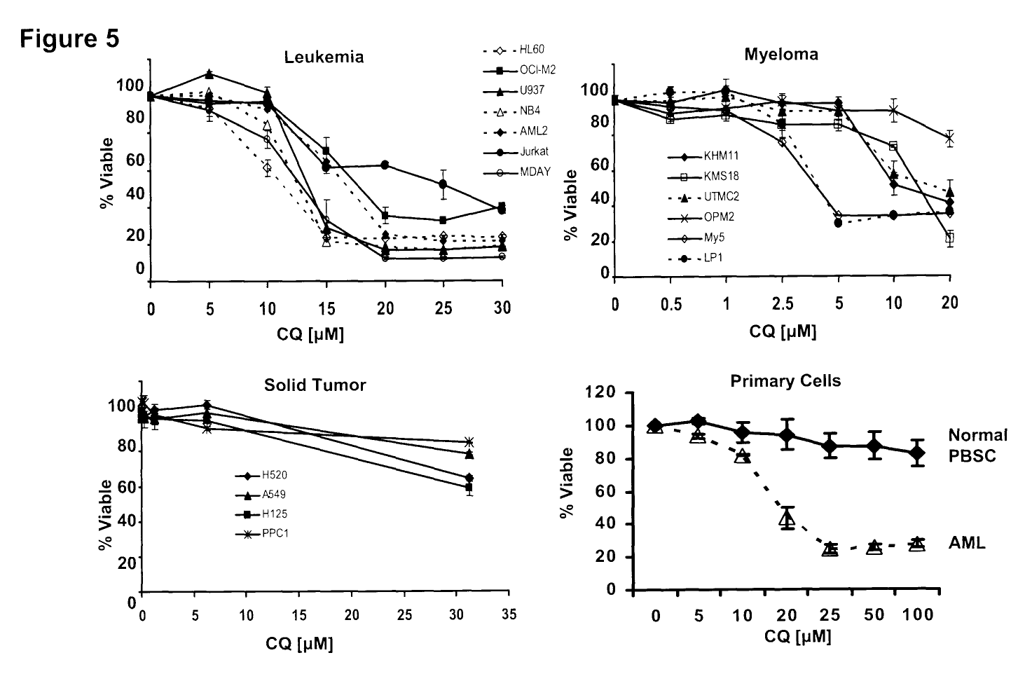

The inventors tested the effects of clioquinol on the viability of acute leukemia (n=7), myeloma (n=14), and solid tumor (n=5) cell lines as well as primary AML samples (n=6) and primary normal hematopoietic cells (n=3). Forty-eight hours after incubation, clioquinol induced cell death in 6/7 AML, 12/14 myeloma, 0/5 solid tumor, 6/7 primary AML patient samples and 0/3 normal hematopoietic cells with an IC50 <20 μM (FIG. 5). Of note, after oral administration of clioquinol, trough serum concentrations of 20 μM can be achieved in patients (32). Reductions in cell viability were associated with clioquinol's ability to inhibit the proteasome, and no proteasomal inhibition was detected in clioquinol-resistant cells, including the normal hematopoietic cells.

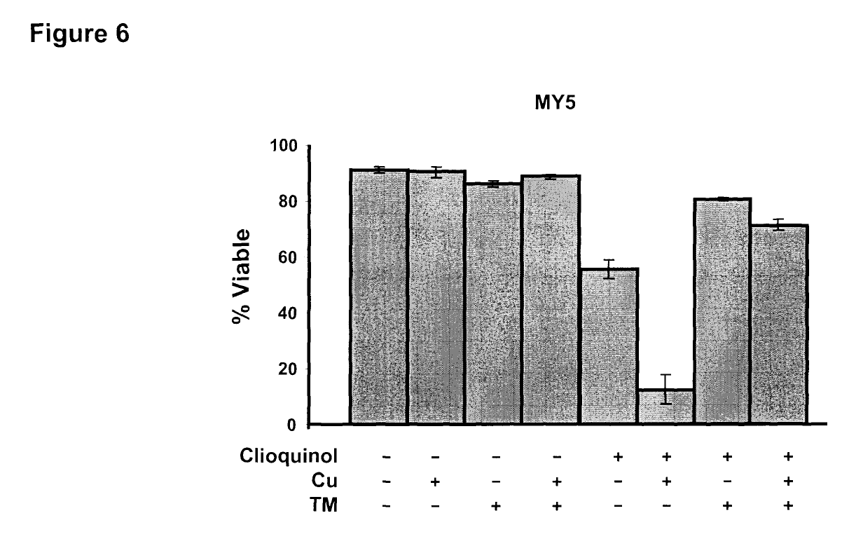

The inventors also demonstrated that clioquinol-induced cell death was copper-dependent as supplementing copper in the medium enhanced the toxicity of clioquinol, while adding the strong copper chelator, tetrathiomoylbdate, abrogated clioquinol-induced cell death (FIG. 6). Like inhibition of the proteasome, supplementing primary AML and normal hematopoietic cells with copper increased the potency of clioquinol.

Thus, these results indicate that clioquinol induces cell death through a copper-dependent mechanism and are consistent with its effects as a copper dependent proteasome inhibitor. ps Clioquinol Delays Tumor Growth in a Xenograft Model of Leukemia

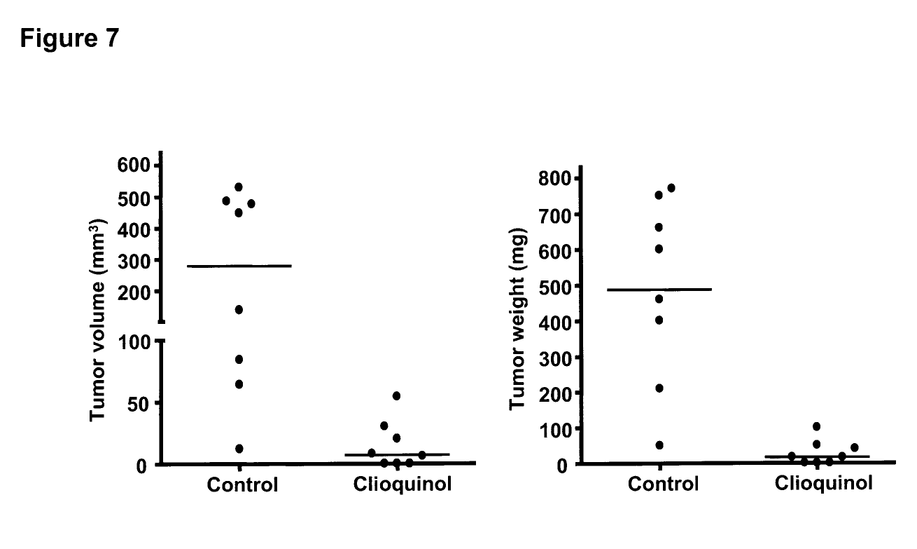

Given the effects on leukemia cell lines and primary patient samples, the inventors evaluated clioquinol in a leukemia xenograft model. MDAY-D2 leukemia cells were injected intraperitoneally into DBA2 mice. Mice were then treated twice daily with oral clioquinol (100 mg/kg) for 10 days. Ten days after treatment, mice were sacrificed and the weight and volume of the intraperitoneal tumor was measured (FIG. 7). Oral clioquinol delayed tumor progression without untoward toxicity or reduction in body weight. Similar results were obtained with a K562 leukemia xenograft. K562 leukemia cells were injected subcutaneous into sublethally irradiated non-scid mice. The clioquinol treatment was initiated when tumors reached volumes of 200 mm³ at which time mice were randomized to receive 100 mg/kg of clioquinol (treated group) or buffer control (untreated group) for 5 of 7 days. Caliper measurements were performed twice weekly to estimate tumor volume and differences compared between treated and untreated groups. Treatment with clioquinol delayed tumor growth in this mouse model.

### Discussion

Acute myeloid leukemia (AML) and multiple myeloma (MM) are malignant diseases resulting in the proliferation of abnormal cells of myeloid and lymphoid origin, respectively. Both diseases are characterized by poor responses to standard therapies. It would be advantageous for these patients and those with relapsed refractory disease if novel therapies were available. As many of these patients are frail, therapies that achieve an anti-myeloma or leukemia effect without significant toxicity are highly desirable.

The high throughput screen identified clioquinol as an inhibitor of cyclin D2 transactivation, which is over-expressed in patients with high risk AML and MM. Subsequently, the inventors demonstrated that this compound induces cell death in myeloma and leukemia cell lines.

### Example 2

### Mouse Xenograft Model

Sublethally irradiated NOD-SCID mice are inoculated subcutaneously in the flanks with U937, LP-1, AND JJN3 cells. The clioquinol treatment is initiated when tumors reach volumes of 200 mm³ at which time mice are randomized to receive 50 mg/kg of clioquinol (treated group) or buffer control (untreated group) for 5 of 7 days. Caliper measurements are performed twice weekly to estimate tumor volume and differences compared between treated and untreated groups.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

1. Goldberg A L. Protein degradation and protection against misfolded or damaged proteins. Nature 2003; 426:895-9.
2. Nalepa G, Rolfe M, and Harper J W. Drug discovery in the ubiquitin-proteasome system. Nat Rev Drug Discov 2006; 5:596-613.
3. Passmore L A and Barford D. Getting into position: the catalytic mechanisms of protein ubiquitylation. Biochem J 2004; 379:513-25.
4. Meister S, Schubert U, Neubert K, et al. Extensive immunoglobulin production sensitizes myeloma cells for proteasome inhibition. Cancer Res 2007; 67:1783-92.
5. Bazzaro M, Lee M K, Zoso A, et al. Ubiquitin-proteasome system stress sensitizes ovarian cancer to proteasome inhibitor-induced apoptosis. Cancer Res 2006; 66:3754-63.
6. Hideshima T, Richardson P, Chauhan D, et al. The proteasome inhibitor PS-341 inhibits growth, induces apoptosis, and overcomes drug resistance in human multiple myeloma cells. Cancer Res 2001; 61:3071-6.
Chauhan D, Catley L, Li G, et al. A novel orally active proteasome inhibitor induces apoptosis in multiple myeloma cells with mechanisms distinct from Bortezomib. Cancer Cell 2005; 8:407-19.
8. Arrigo A P, Tanaka K, Goldberg A L, and Welch W J. Identity of the 19S ‘prosome’ particle with the large multifunctional protease complex of mammalian cells (the proteasome). Nature 1988; 331:192-4.
9. Kopp F and Kuehn L. Orientation of the 19S regulator relative to the 20S core proteasome: an immunoelectron microscopic study. J Mol Biol 2003; 329:9-14.
10. Seemuller E, Lupas A, Stock D, Lowe J, Huber R, and Baumeister W. Proteasome from Thermoplasma acidophilum: a threonine protease. Science 1995; 268:579-82.
11. Lowe J, Stock D, Jap B, Zwickl P, Baumeister W, and Huber R. Crystal structure of the 20S proteasome from the archaeon T. acidophilum at 3.4 A resolution. Science 1995; 268:533-9.
12. Kisselev A F, Akopian T N, Castillo V, and Goldberg A L. Proteasome active sites allosterically regulate each other, suggesting a cyclical bite-chew mechanism for protein breakdown. Mol Cell 1999; 4:395-402.
13. Williamson M J, Blank J L, Bruzzese F J, et al. Comparison of biochemical and biological effects of ML858 (salinosporamide A) and bortezomib. Mol Cancer Ther 2006; 5:3052-61.
14. Groll M, Huber R, and Potts B C. Crystal structures of Salinosporamide A (NPI-0052) and B (NPI-0047) in complex with the 20S proteasome reveal important consequences of beta-lactone ring opening and a mechanism for irreversible binding. J Am Chem Soc 2006; 128:5136-41.
15. Groll M, Berkers C R, Ploegh H L, and Ovaa H. Crystal structure of the boronic acid-based proteasome inhibitor bortezomib in complex with the yeast 20S proteasome. Structure 2006; 14:451-6.
16. Richardson P G, Sonneveld P, Schuster M W, et al. Bortezomib or high-dose dexamethasone for relapsed multiple myeloma. N Engl J Med 2005; 352:2487-98.
17. Capel I D, Pinnock M H, Williams D C, and Hanham I W. The serum levels of some trace and bulk elements in cancer patients. Oncology 1982; 39:38-41.
18. Carpentieri U, Myers J, Thorpe L, Daeschner C W, 3rd, and Haggard M E. Copper, zinc, and iron in normal and leukemic lymphocytes from children. Cancer Res 1986; 46:981-4.
19. Zuo X L, Chen J M, Zhou X, Li X Z, and Mei G Y. Levels of selenium, zinc, copper, and antioxidant enzyme activity in patients with leukemia. Biol Trace Elem Res 2006; 114:41-53.
20. Carter B Z, Gronda M, Wang Z, et al. Small-molecule XIAP inhibitors derepress downstream effector caspases and induce apoptosis of acute myeloid leukemia cells. Blood 2005; 105:4043-50.
21. Yata K, Sadahira Y, Otsuki T, et al. Cell cycle analysis and expression of cell cycle regulator genes in myeloma cells overexpressing cyclin D1. Br J Haematol 2001; 114:591-9.
22. Ely S, Di Liberto M, Niesvizky R, et al. Mutually exclusive cyclin-dependent kinase 4/cyclin D1 and cyclin-dependent kinase 6/cyclin D2 pairing inactivates retinoblastoma protein and promotes cell cycle dysregulation in multiple myeloma. Cancer Res 2005; 65:11345-53.
23. Bergsagel P L, Kuehl W M, Zhan F, Sawyer J, Barlogie B, and Shaughnessy Jr J. Cyclin D dysregulation: an early and unifying pathogenic event in multiple myeloma. Blood 2005.
24. Hurt E M, Wiestner A, Rosenwald A, et al. Overexpression of c-maf is a frequent oncogenic event in multiple myeloma that promotes proliferation and pathological interactions with bone marrow stroma. Cancer Cell 2004; 5:191-9.
25. An W G, Hwang S G, Trepel J B, and Blagosklonny M V. Protease inhibitor-induced apoptosis: accumulation of wt p53, p21WAF1/CIP1, and induction of apoptosis are independent markers of proteasome inhibition. Leukemia 2000; 14:1276-83.
26. Bogner C, Schneller F, Hipp S, Ringshausen I, Peschel C, and Decker T. Cycling B-CLL cells are highly susceptible to inhibition of the proteasome: involvement of p27, early D-type cyclins, Bax, and caspase-dependent and -independent pathways. Exp Hematol 2003; 31:218-25.
27. Chen W J and Lin J K. Induction of G1 arrest and apoptosis in human jurkat T cells by pentagalloylglucose through inhibiting proteasome activity and elevating p27Kip1, p21Cip1/WAF1, and Bax proteins. J Biol Chem 2004; 279:13496-505.
28. Ding W Q, Liu B, Vaught J L, Yamauchi H, and Lind S E. Anticancer activity of the antibiotic Clioquinol. Cancer Res 2005; 65:3389-95.
29. Daniel K G, Chen D, Orlu S, Cui Q C, Miller F R, and Dou Q P. Clioquinol and pyrrolidine dithiocarbamate complex with copper to form proteasome inhibitors and apoptosis inducers in human breast cancer cells. Breast Cancer Res 2005; 7:R897-908.
30. Chen D, Cui Q C, Yang H, et al. Clioquinol, a therapeutic agent for Alzheimer's disease, has proteasome-inhibitory, androgen receptor-suppressing, apoptosis-inducing, and antitumor activities in human prostate cancer cells and xenografts. Cancer Res 2007; 67:1636-44.
31. Davison A J, Kettle A J, and Fatur D J. Mechanism of the inhibition of catalase by ascorbate. Roles of active oxygen species, copper and semidehydroascorbate. J Biol Chem 1986; 261:1193-200.
32. Cherny R A, Atwood C S, Xilinas M E, et al. Treatment with a copper-zinc chelator markedly and rapidly inhibits beta-amyloid accumulation in Alzheimer's disease transgenic mice. Neuron 2001; 30:665-76.

1. A method of treating a hematological malignancy comprising administering an effective amount of clioquinol to a subject in need of such treatment wherein the hematological malignancy is multiple myeloma, acute myeloid leukemia or high-risk acute myeloid leukemia.

2-5. (canceled)

6. The method of claim 1 wherein the hematological malignancy is a refractory malignancy.

7-10. (canceled)

11. The method of claim 1 further comprising contemporaneously administering a copper compound.

12. The method of claim 1, wherein said effective amount is within the range of about 1 to about 200 mg/kg body weight or within the range of about 5 to about 50 mg/kg body weight.

13. (canceled)

14. A pharmaceutical composition for treatment of a hematological malignancy comprising an effective amount of clioquinol and a pharmaceutically acceptable carrier in a dosage form, wherein the hematological malignancy is multiple myeloma, acute myeloid leukemia or high-risk acute myeloid leukemia.

15. The pharmaceutical composition of claim 14, wherein said dosage form is suitable for oral administration.

16. The pharmaceutical composition of claim 14, wherein said dosage form is suitable for injection.

17. The pharmaceutical composition of claim 15, wherein said dosage form is a solid dosage form that contains from about 20 mg to about 1000 mg of said clioquinol.

18. The pharmaceutical composition of claim 15, wherein said dosage form is a solid dosage form that contains from about 50 mg to about 500 mg of said clioquinol.

19. The pharmaceutical composition of claim 14, wherein said dosage form is a liquid dosage form that contains from about 20 mg to about 2000 mg of said clioquinol.

20. The pharmaceutical composition of claim 14, wherein said dosage form is a liquid dosage form that contains from about 40 mg to about 500 mg of said clioquinol.

21. The pharmaceutical composition of claim 14 for treatment of multiple myeloma, acute myeloid leukemia or high-risk acute myeloid leukemia in a subject, which composition comprises as active ingredient clioquinol and a pharmaceutically acceptable carrier in unit dosage form.

22. The pharmaceutical composition of claim 21, which is suitable for oral administration or injection.

23. (canceled)

24. The pharmaceutical composition of claim 14 for treatment of a hematological malignancy comprising clioquinol and a pharmaceutically acceptable carrier in unit dosage form in an amount suitable to provide about 1 to about 200 mg of clioquinol/kg body weight formulated into a solid oral dosage form, a liquid dosage form, or an injectable dosage form, wherein the hematological malignancy is multiple myeloma, acute myeloid leukemia or high-risk acute myeloid leukemia.

25. The pharmaceutical composition of claim 14 for treatment of a hematological malignancy comprising clioquinol and a pharmaceutically acceptable carrier in unit dosage form in an amount suitable to provide about 5 to about 50 mg of clioquinol/kg body weight formulated into a solid oral dosage form, a liquid dosage form, or an injectable dosage form, wherein the hematological malignancy is multiple myeloma, acute myeloid leukemia or high-risk acute myeloid leukemia.

26. (canceled)

27. The pharmaceutical composition of claim 14 wherein the oral dosage form is selected from enteric coated tablets, caplets, gelcaps, and capsules.

28-31. (canceled)

32. A commercial package comprising a composition according to claims 14, and associated therewith instructions for the use thereof for treatment of multiple myeloma, acute myeloid leukemia or high-risk acute myeloid leukemia in a subject in need of such treatment.

33. The method of claim 1 wherein the subject is administered a pharmaceutical composition comprising an effective amount of clioquinol and a pharmaceutically acceptable carrier in a dosage form.

34-45. (canceled)

46. The method of claim 33 wherein said dosage form is suitable for oral administration or injection.

47. The method of claim 46 wherein said dosage is a solid dosage form that contains from about 20 mg to about 2000 mg of said clioquniol or said dosage form is a liquid dosage form that contains from about 20 mg to about 2000 mg of said clioquinol.
